SEQUENCE LISTING (35 sequences)

SEQ ID NO: 1 - protein, 5 aa, Artificial
  A-motif
XXDYS

SEQ ID NO: 2 - protein, 5 aa, Artificial
  B-motif
GXPSG

SEQ ID NO: 3 - protein, 4 aa, Artificial
  C-motif
YGDD

SEQ ID NO: 4 - protein, 5 aa, Artificial
  D-motif
XXYGL

SEQ ID NO: 5 - protein, 10 aa, Artificial
  E-Motif
XXXXFLXRXX

SEQ ID NO: 6 - protein, 520 aa, Norovirus
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALE

SEQ ID NO: 7 - protein, 517 aa, Sapovirus
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 8 - protein, 533 aa, Vesivirus
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLE

SEQ ID NO: 9 - protein, 517 aa, Lagovirus
MTSNFFCGEPIDYRGITAHRLVGAEPRPPVSGTRYAKVPGVPDEYKTGYRPANLGRSDPDSDKSLMNIAVKNLQVYQQEPKLDKVDEFIERAAADVLGYLRFLTKGERQANLNFKAAFNTLDLSTSCGPFVPGKKIDHVKDGVMDQVLAKHLYKCWSVANSGKALHHIYACGLKDELRPLDKVKEGKKRLLWGCDVGVAVCAAAVFHNICYKLKMVARFGPIAVGVDMTSRDVDVIINNLTSKASDFLCLDYSKWDSTMSPCVVRLAIDILADCCEQTELTKSVVLTLKSHPMTILDAMIVQTKRGLPSGMPFTSVINSICHWLLWSAAVYKSCAEIGLHCSNLYEDAPFYTYGDDGVYAMTPMMVSLLPAIIENLRDYGLSPTAADKTEFIDVCPLNKISFLKRTFELTDIGWVSKLDKSSILRQLEWSKTTSRHMVIEETYDLAKEERGVQLEELQVAAAAHGQEFFNFVCRELERQQAYTQFSVYSYDAARKILADRKRVVSVVPDDEFVNVME

SEQ ID NO: 10 - protein, 526 aa, Artificial
  Norovirus RNA polymerase containing His tag
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALEHHHHHH

SEQ ID NO: 11 - protein, 523 aa, Artificial
  Sapovirus RNA polymerase containing His tag
MKHHHHHHDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 12 - protein, 523 aa, Artificial
  Sapovirus RNA polymerase containing His tag
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEMEHHHHHH

SEQ ID NO: 13 - protein, 539 aa, Artificial
  Vesivirus RNA polymerase containing His tag
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLEHHHHHH

SEQ ID NO: 14 - protein, 523 aa, Artificial
  Lagovirus RNA Polymerase containing His tag
MTSNFFCGEPIDYRGITAHRLVGAEPRPPVSGTRYAKVPGVPDEYKTGYRPANLGRSDPDSDKSLMNIAVKNLQVYQQEPKLDKVDEFIERAAADVLGYLRFLTKGERQANLNFKAAFNTLDLSTSCGPFVPGKKIDHVKDGVMDQVLAKHLYKCWSVANSGKALHHIYACGLKDELRPLDKVKEGKKRLLWGCDVGVAVCAAAVFHNICYKLKMVARFGPIAVGVDMTSRDVDVIINNLTSKASDFLCLDYSKWDSTMSPCVVRLAIDILADCCEQTELTKSVVLTLKSHPMTILDAMIVQTKRGLPSGMPFTSVINSICHWLLWSAAVYKSCAEIGLHCSNLYEDAPFYTYGDDGVYAMTPMMVSLLPAIIENLRDYGLSPTAADKTEFIDVCPLNKISFLKRTFELTDIGWVSKLDKSSILRQLEWSKTTSRHMVIEETYDLAKEERGVQLEELQVAAAAHGQEFFNFVCRELERQQAYTQFSVYSYDAARKILADRKRVVSVVPDDEFVNVMEHHHHHH

SEQ ID NO: 15 - DNA, 24 bp, Artificial
  ssDNA template
atacctagaatctgaccaaccccc

SEQ ID NO: 16 - DNA, 19 bp, Artificial
  ssDNA template
atacctagaatctgaccaa

SEQ ID NO: 17 - DNA, 24 bp, Artificial
  ssDNA template having 5 rC at the 3′ end
atacctagaatctgaccaaccccc

SEQ ID NO: 18 - RNA, 24 nt, Artificial
  ssRNA template
auaccuagaaucugaccaaccccc

SEQ ID NO: 19 - DNA, 74 bp, Artificial
  ssDNA template
atacctagaatgtgaccaaatacctagaatctgaccaacgaaaaaaaaaataagcacgaagctcagagtccccc

SEQ ID NO: 20 - DNA, 10 bp, Artificial
  DNA oligonucleotide primer
ttttactgga

SEQ ID NO: 21 - DNA, 24 bp, Artificial
  ssDNA template
cccccttggtcagattctaggtat

SEQ ID NO: 22 - DNA, 25 bp, Artificial
  ssDNA template having one rC nucleotide at the 3′-end
cccccttggtcagattctaggtatc

SEQ ID NO: 23 - DNA, 24 bp, Artificial
  ssRNA template having 5 dC at the 3′-end
uaagcacgaagcucagaguccccc

SEQ ID NO: 24 - RNA, 24 nt, Artificial
  ssRNA template
uaagcacgaagcucagaguccccc

SEQ ID NO: 25 - DNA, 30 bp, Artificial
  template containing antisense sequence of siRNA and half recognition sequence of Bsr I and 5 dC at the 3′-end
auaccuagaaucugaccaatccagtccccc

SEQ ID NO: 26 - DNA, 11 bp, Artificial
  ssDNA primer
gggggactgga

SEQ ID NO: 27 - DNA, 30 bp, Artificial
  complementary RNA strand (containing primer sequence) to template containing antisense sequence of siRNA and half recognition sequence of Bsr I and 5 dC at the 3′-end
gggggactggauuggucagauucuagguau

SEQ ID NO: 28 - DNA, 21 bp, Artificial
  antisense or “guide” strand of siRNA having 3′-overhang at the antisense or “guide” strand
auaccuagaaucugaccaatc

SEQ ID NO: 29 - DNA, 20 bp, Artificial
  sense or “passenger” strand of siRNA having 3′-overhang at the antisense or “guide” strand
auuggucagauucuagguau

SEQ ID NO: 30 - RNA, 11 nt, Artificial
  short complementary RNA sequence after de novo initiation of RNA polymerisation
gggggacugga

SEQ ID NO: 31 - RNA, 30 nt, Artificial
  Complementary RNA strand to template containing antisense sequence of siRNA and half recognition sequence of Bsr I and 5 dC at the 3′-end
gggggacuggauuggucagauucuagguau

SEQ ID NO: 32 - DNA, 40 bp, Artificial
  template containing antisense sequence of siRNA and half recognition sequence of Bsr I and 5 dC at the 3′-end and half recognition sequence of BsrD I at the 5′-end
ttgcaatgaaauaccuagaaucugaccaatccagtccccc

SEQ ID NO: 33 - DNA, 40 bp, Artificial
  complementary RNA strand (containing primer sequence) to template containing antisense sequence of siRNA and half recognition sequence of Bsr I and 5 dC at the 3′-end and half recognition sequence of BsrD I at the 5′-end
gggggactggauuggucagauucuagguauuucauugcaa

SEQ ID NO: 34 - DNA, 22 bp, Artificial
  sense or “passenger” strand of siRNA having 3′-overhang at antisense or “guide” strand and having 3′-overhang at sense or “passenger” strand
auuggucagauucuagguauuu

SEQ ID NO: 35 - RNA, 40 nt, Artificial
  Complementary RNA strand to template containing antisense sequence of siRNA and half recognition sequence of Bsr I and 5 dC at the 3′-end and half recognition sequence of BsrD I at the 5′-end
gggggacuggauuggucagauucuagguauuucauugcaa

(19) United States
(12) Patent Application Publication
(10) Pub. No.: US 2013/0183718 A1
(43) Pub. Date: Jul. 18, 2013

(54) Method for Synthesizing RNA using DNA Template

(71) Applicant: Jacques Rohayem, Dresden (DE)

(72) Inventor: Jacques Rohayem, Dresden (DE)

(73) Assignee: RibpxX GmbH, Radebuelde (DE)

(21) Appl. No.: 13/825,112

(22) PCT Filed: Sep. 20, 2011
(86) PCT No.: PCT/EP2011/066360

§ 371 Date: Mar. 19, 2013

(30) Foreign Application Priority Data
Sep. 21, 2010 (EP) 10178113.6

(51) Int. Cl. C12P 19/34

(52) U.S. Cl. CPC C12P 19/34
USPC 435/91.3; 435/91.5; 435/91.53; 435/194

(57) ABSTRACT

The present invention relates to a method of RNA synthesis by RNA-dependent RNA polymerases (RdRp) displaying an RNA polymerase activity on single-stranded DNA templates and to a kit for carrying out the method. The RdRp showing DNA-dependent RNA polymerase activity has a “right hand conformation” and the amino acid sequence of said RdRp comprises a conserved arrangement of the following sequence motifs: a. XXDYS, b. GXPSG, c. YGDD, d. XXYGL, e. XXXXFLXRXX (with the following meanings: D: aspartate, Y: tyrosine, S: serine, G: glycine, P: proline, L: leucine, F: phenylalanine, R: arginine, X: any amino acid). This class of RdRp is exemplified by the RdRp enzymes of viruses of the Caliciviridae family. The present invention also relates to a method for transferring at least one ribonucleotide (rC, rA, rU or rG) to the 3′-end of a single-stranded DNA by using the RdRp of the invention.

The present invention relates to a method of RNA synthesis by RNA-dependent RNA polymerases (RdRp) displaying an RNA polymerase activity on single-stranded DNA templates and to a kit for carrying out the method. The RdRp showing DNA-dependent RNA polymerase activity has a “right hand conformation” and the amino acid sequence of said RdRp comprises a conserved arrangement of the following sequence motifs: a. XXDYS (SEQ ID NO: 1), b. GXPSG (SEQ ID NO: 2), c. YGDD (SEQ ID NO: 3), d. XXYGL (SEQ ID NO: 4), e. XXXXFLXRXX (SEQ ID NO: 5) (with the following meanings: D: aspartate, Y: tyrosine, S: serine, G: glycine, P: proline, L: leucine, F: phenylalanine, R: arginine, X: any amino acid). This class of RdRps is exemplified by the RdRp enzymes of viruses of the Caliciviridae family. The present invention also relates to a method for transferring at least one ribonucleotide (rC, rA, U or rG) to the 3′-end of a single-stranded DNA by using the RdRp of the invention.

RdRps of use in the present invention are known from viruses such as those of the Caliciviridae family having a single-stranded RNA (ssRNA) of positive polarity as the viral genome (see, e.g., Rohayem et al. Antiviral Research, 87 (2010): 162-178). RdRps of this type have been shown to be useful for primer-dependent and independent amplification of RNA and also show a terminal transferase acitivity on RNA templates (see WO-A-2007/012329). Primer-independent RNA synthesis on single-stranded templates is especially useful in the context of providing short dsRNA molecules for siRNA applications (see WO-A-2007/012329). Furthermore, such enzymes have been shown to be capable of employing modified ribonucleotides when synthesizing an RNA strand complementary to an ssRNA template (see WO-A-2009/150156).

Since ssRNA templates are (i) expensive in comparison to single-stranded DNA (ssDNA), if produced by chemical synthesis and (ii) are much more sensitive in comparison to DNA with respect degradation, it would be desirable to have means for providing RNA synthesized on DNA templates.

Conventional DNA-dependent RNA polymerases require specific promoter sequences for initiation of RNA polymerisation (for a recent review, see, for example, Temiakov et al., Cell 2004 (116): 381-391).

The technical problem underlying the present invention is therefore the provision of simple means for transcribing DNA sequences into RNA.

The solution to the above technical problem is provided by the embodiments of the present invention as described herein and characterised in the claims.

The present invention is based on the surprising finding that RdRps having the structural features as outlined herein are capable of synthesizing a complementary strand on single-stranded DNA templates, i.e. show a DNA-dependent RNA polymerase activity. Furthermore, the RdRps as described herein show a terminal transferase activity on ssDNA templates, i.e. add one or more ribonucleotides to the 3′-end of single-stranded DNA.

Thus, according to a first aspect, the present invention provides a method for transcribing a single-stranded polynucleotide template containing at least a segment of DNA into complementary RNA comprising the step of incubating said template with an RNA-dependent RNA polymerase (RdRp) having DNA-dependent RNA polymerase activity in the presence or absence of a primer hybridised to the single-stranded template under conditions such that said RdRp synthesizes an RNA strand complementary to said template producing a double-stranded molecule comprising at least a segment of hybrid DNA/RNA, wherein the RdRp having DNA-dependent RNA polymerase activity has a “right hand conformation” and the amino acid sequence of said RdRp comprises a conserved arrangement of the following sequence motifs:

a.
(SEQ ID NO: 1)
XXDYS
b.
(SEQ ID NO: 2)
GXPSG
c.
(SEQ ID NO: 3)
YGDD
d.
(SEQ ID NO: 4)
XXYGL
e.
(SEQ ID NO: 5)
XXXXFLXRXX

with the following meanings:

D: aspartate

Y: tyrosine

S: serine

G: glycine

P: proline

L: leucine

F: phenylalanine

R: arginine

X: any amino acid.

The so-called “right hand conformation” as used herein means that the tertiary structure (conformation) of the protein folds like a right hand with finger, palm and thumb, as observed in most template-dependent polymerases.

The sequence motif “XXDYS” (SEQ ID NO: 1) is the so-called A-motif. The A-motif is responsible for the discrimination between ribonucleosides and deoxyribonucleosides. The motif “GXPSG” (SEQ ID NO: 2) is the so-called B-motif. The B-motif is conserved within all representatives of this RdRp family of the corresponding polymerases from the Caliciviridae. The motif “YGDD” (C-motif, SEQ ID NO: 3) represents the active site of the enzyme. This motif, in particular the first aspartate residue (in bold, YGDD) plays an important role in the coordination of the metal ions during the Mg²⁺/Mn²⁺ dependent catalysis. The motif “XXYGL” (SEQ ID NO: 4) is the so-called D-motif. The D-motif is a feature of template-dependent polymerases. Finally, the “XXXXFLXRXX” motif (E-motif, SEQ ID NO: 5) is a feature of RNA-dependent RNA polymerases which discriminates them from (exclusively) DNA-dependent RNA polymerases.

Typical representatives of the above types of RdRps are the corresponding enzymes of the calicivirus family (Caliciviridae). Preferably, the RdRp having DNA-dependent RNA polymerase activity is an RdRp of a human and/or non-human pathogenic calicivirus. Especially preferred is an RdRp of a norovirus, sapovirus, vesivirus or lagovirus, for example the RdRP of the norovirus strain HuCV/NL/Dresden174/1997/GE (GenBank Acc. No AY741811) or an RdRp of the sapovirus strain pJG-Sap01 (GenBank Acc. No AY694184) or an RdRp of the vesivirus strain FCV/Dresden/2006/GE (GenBank Acc. No DQ424892) or an RdRp of the lagovirus strain pJG-RHDV-DD06 (GenBank Acc. No. EF363035.1).

According to especially preferred embodiments of the invention the RdRp having DNA-dependent RNA polymerase activity is a protein comprising (or having) an amino acid sequence according SEQ ID NO: 6 (norovirus RdRp), SEQ ID NO: 7 (sapovirus RdRp), SEQ ID NO: 8 (vesivirus RdRp) or SEQ ID NO: 9 (lagovirus RdRp). The person skilled in the art is readily capable of preparing such RdRp, for example by recombinant expression using suitable expression vectors and host organisms (cf. WO-A-2007/012329). To facilitate purification of the RdRp in recombinant expression, it is preferred that the RdRp is expressed with a suitable tag (for example GST or (His)₆-tag) at the N- or C-terminus of the corresponding sequence. For example, a histidine tag allows the purification of the protein by affinity chromatography over a nickel or cobalt column in a known fashion. Examples of embodiments of RdRps fused to a histidine tag are the proteins comprising (or having) an amino acid sequence according to SEQ ID NO: 10, SEQ ID NO: 11, SEQ ID NO: 12, SEQ ID NO: 13 or SEQ ID NO: 14. SEQ NO: NO: 10 corresponds to a norovirus RdRp having a histidine tag. SEQ ID NO: 11 and SEQ ID NO: 12 correspond to amino acid sequences of a sapovirus RdRps having a histidine tag. SEQ ID NO: 13 corresponds to the amino acid sequence of a vesirius RdRp having a histidine tag. SEQ ID NO: 14 corresponds to the amino acid sequence of a lagovirus RdRp having a histidine tag.

In contrast to other RNA-dependent RNA polymerases, e.g. RNA-dependent RNA polymerases such as replicases of the Qβ type, the RdRps as defined herein do not require primers having a specific recognition sequence for the polymerase to start RNA synthesis. Thus, a “primer” as used herein is typically a primer not having such recognition sequences, in particular, of RNA polymerases. Furthermore, the RdRps of use in the present invention are different from usual DNA-dependent RNA polymerases such as T7 RNA polymerase in that they do not require specific promoter sequences to be present in the template.

The above-defined RdRp having DNA-dependent RNA polymerase activity is capable of synthesizing a complementary RNA strand on a polynucleotide strand consisting of or at least comprising one or more DNA segments both by elongation of a primer with a complementary sequence to a partial sequence of the template DNA and by de novo synthesis of a complementary strand in the absence of a primer. However, if the polynucleotide template has a deoxy-T, deoxy-A or deoxy-G nucleotide at its 3′-end (i.e. the last nucleotide at the 3′-end of the single-stranded template), the RdRp having DNA-dependent RNA polymerase activity useful in the present invention requires the presence of a primer hybridised to the template for synthesis of an RNA strand complementary to the template. If the polynucleotide template as defined herein consists of or contains one or more deoxyribonucleotides at its 3′-end (i.e. the 3′-end of the template is a DNA segment or only the last nucleotide is a deoxyribonucleotide), it is preferred that the last deoxyribonucleotide at the 3′-end of the template is a dC, more preferred at least the last two, three, four or five deoxyribonucleotides at the 3′-end of the template are dC nucleotides for efficient de novo initiation of RNA synthesis in the absence of a primer.

The primer, if desired or required, respectively, may be a sequence specific (heteropolymeric) DNA or RNA or mixed DNA/RNA primer or may be a random primer (DNA or RNA or mixed DNA/RNA) or may be a homopolymeric primer such as an oligo-dT-primer or an oligo-U-Primer. The length of the primer is not critical for carrying out the inventive method, but usually oligonucleotide primers having a length of, for example, about 5 to about 25 nt, more preferred about 10 to 20 nt, most preferred about 15 to about 20 nt, are especially useful. More details of the characteristic features of the calicivirus RdRp can be found in WO-A-2007/012329.

The single-stranded polynucleotide template of the present invention comprises at least a sequence segment of deoxyribonucleotides, i.e. at least a segment of ssDNA, e.g. at least a segment of DNA at the 3′-end of the template. A “segment” in this context means at least 2 or more consecutive deoxyribonucleotides. For example, the polynucleotide template according to the invention may be a single-stranded molecule starting at its 5′-end with ribonucleotides, followed by a “middle” region of deoxyribonucleotides (DNA) and ends (at the 3′-end) again with ribonucleotides. Other examples are species of 5′-RNA-DNA-3′ or 5′-DNA-RNA-3′ or any other polynucleotides having RNA and DNA sequences. Further examples of single-stranded polynucleotide templates according to the invention include species of predominantly ssDNA, but having one to multiple (such as 1, 2, 3, 4, 5, 6, 7, 8, 9 or 10) ribonucleotides at one or both of the 5′-end and/or 3′-end, preferably at the 3′-end. Alternatively, templates of use according to the invention may be predominantly ssRNA, but having one to multiple (such as 1, 2, 3, 4, 5, 6, 7, 8, 9 or 10) deoxyribonucleotides at one or both of the 5′-end and/or 3′-end, preferably at the 3′-end. Of course, the single-stranded polynucleotide template according to the invention may also consist exclusively of ssDNA or ssRNA.

As mentioned before, polynucleotide templates of the invention having a dA, dT or dG residue at the 3′-end normally require a primer for synthesis of a complementary RNA strand by the RdRp having DNA-dependent RNA polymerase activity. However, even such polynucleotide sequences not having a C nucleotide at the 3′-end can be efficiently transcribed into RNA by the inventive method without the need of a primer: in this case the method may be carried with an initial step of incubating the single-stranded polynucleotide template with the RdRp as defined above in the presence of rCTP as the only nucleotide under conditions such that said RdRp adds at least one rC (or more such as 2, 3, 4 or 5 rC) nucleotide to the 3′-end of the ssDNA or ssDNA segment, respectively. Thereafter, the thus produced template having one or more C ribonucleotides at the 3′-end can be introduced to the step of incubation with the RdRp such that said RdRp synthesizes a complementary RNA strand, which step may be carried out in the absence of a primer. It is to be understood, however, that also a primer may be used in this embodiment, for example, if needed to introduce a chosen sequence into the RNA strand to be produced by the RdRp or for other purposes.

According to a preferred embodiment of the method according to the invention, the double-stranded molecule (polynucleotide) comprising at least a segment of hybrid DNA/RNA is separated into single strands resulting in an ssRNA and the template, i.e. a single-stranded molecule comprising at least a segment of ssDNA (this step will be in the following denoted as “step (b)”). This step may be carried out by heat or microwave irradiation or chemical denaturation or enzymatically, e.g. by an enzyme capable of separating single-stranded polynucleotides into single-stranded ones such as a helicase. In an especially preferred embodiment of the present invention this and other separation steps of double-stranded polynucleotides produced by the RdRp as defined herein is carried out by the same enzyme, i.e. the RdRp itself. This step makes beneficial use of the strand-displacement activity of the RdRps as defined herein.

It is further preferred that the single strands obtained in step (b) (i.e. the ssRNA and the single-stranded molecule comprising at least a segment of ssDNA, e.g. single-stranded molecule being purely ssDNA) are again incubated with the RdRp as defined herein under conditions such that the RdRp synthesizes an RNA strand complementary to each of said single strands to form double-stranded RNA (dsRNA) and a double-stranded molecule comprising at least a segment of hybrid DNA/RNA (in the following denoted as “step (c)”). Preferably, a further strand separation step follows (“step (d)”). It is evident that the steps of RNA synthesis (c) and strand separation (d) can be repeated one or more times (“step (e)”), e.g. about 3 to about 40, preferably about 5 to about 30, more preferably about 10 to about 20 times. In case the steps of strand separation and RNA synthesis are carried out several times, it is clear that dsRNA species accumulate over species having an RNA strand and a strand being DNA (or at least having a segment of DNA). According to a further preferred embodiment, the transcription method of the invention comprises a final RNA synthesis step. In particular in cases of repeated cycling of strand separation and RNA synthesis, this method leads to the production of almost pure dsRNA.

As in the case of step (b) any further strand separation step (d) may be carried out by heat or microwave irradiation or chemical denaturation or enzymatically, e.g. by an enzyme capable of separating single-stranded polynucleotides into single-stranded ones such as a helicase. More preferably, however, step (d) is also carried out by the RdRp as defined herein for enzymatic strand separation. Therefore, it is evident that the preferred method according to the invention comprising several to a multitude of strand separation and RNA synthesis steps may be carried out in a single batch reaction (especially when using templates that do not require a primer for RNA synthesis by the RdRp as defined herein) requiring only one incubation of a reaction mixture containing the template, RdRp, appropriate buffer (see below) and rNTPs (i.e. rATP, rUTP, rCTP and rGTP, or modified rNTPs as further outlined below) for an appropriate period of time such as about 30 min to about 2 h, e.g. about 1 h, at an appropriate temperature such as about 28 to about 42° C., e.g. about 30° C. Alternatively, microwave irradiation can be used, e.g. 50 to 1000 Watts for 5 to 60 seconds.

According to the present invention, the term “conditions such that the RdRp synthesis an RNA strand complementary to the template” means the conditions, in particular relating to buffer, temperature, salt and metal ion (if applicable) conditions that allow the RdRp to synthesise an RNA strand complementary to a template strand. Appropriate buffer, salt, metal ion, reducing agent (if applicable) and other conditions of RdRps are known to the skilled person. With regard to the RdRPs of caliciviruses, it is referred to WO-A-2007/012329. Thus, the ssRNA template is used in amounts of, e.g. 1 microgram to 4 microgram per 50 microliter reaction volume. The concentration of the ribonucleoside triphosphates (including optional modified ribonucleoside trisphosphate(s) as further outlined below) is preferably in the range of from 0.1 micromol/Ito 1micromol/l, for example 0.4 micromol/l. The concentration of the RdRp may be for example 1 micromol/l to 10 micromol/l.

Typical buffer conditions are 10 to 80 mM, more preferred 20 to 50 mM HEPES, pH 7.0-8.0, 1 to 5 mM, for example 3 mM magnesium acetate, magnesium chloride, manganese acetate or manganese chloride and 1 to 5 mM of a reducing agent, for example DTT.

A typical stop solution contains 2 to 10 mM, preferably 4 to 8 mM ammonium acetate, and 50 to 200 mM, for example 150 mM EDTA.

It is further contemplated that the RdRp employs modified ribonucleotides during RNA synthesis. For example, the modification may be a label for detecting the double-stranded RNA synthesis product of the RdRp. Alternatively, also the labelling may carried out for detection of the ssRNA product obtained after strand separation. Labels of use in the present invention comprise fluorophores (such fluoresceine), radioactive groups (e.g. ³²P-labelled ribonucleotides) and partners of specific binding pairs such as biotinylated rNTPs.

The length and origin of the single-stranded polynucleotide, e.g. an ssDNA template, is generally not critical. The template may have a naturally occurring or artificial sequence, and the ssDNA-containing template may be chemically synthesized or derived from diverse sources such as genomic DNA from eukaryotic, prokaryotic or viral origin, plasmid DNA, cDNA, bacmids or any other sources of DNA. Double-stranded DNA needs to be separated into ssDNA by heat or microwave irradiation or chemical denaturation prior to serving a template in the method of the invention.

The method of the present invention is particularly useful for providing short RNA molecules for gene silencing applications, either by antisense technology or RNA interference, also for antisense directed against defined sequences of microRNA or non-coding RNA with the aim to inhibit microRNA-driven RNA interference (antagomirs).

For such applications, the DNA template to be used in the method of the present invention has typically a length of 8 to 45 nucleotides such as of 15 to 30 nucleotides, preferably of 21 to 28 nucleotides, more preferably of 21 to 23 nucleotides. The molecules of the latter length are particularly useful for siRNA applications.

In certain embodiments of the invention, the at least one modified ribonucleotide to be incorporated by the RdRp activity into the complementary strand may have a chemical modification (one or more of them) at the ribose, phosphate and/or base moiety. With respect to molecules having an increased stability, especially with respect to RNA degrading enzymes, modifications at the backbone, i.e. the ribose and/or phosphate moieties, are especially preferred.

The chemically modified RNA products of the methods of the present invention preferably have an increased stability as compared to the non-modified ss- of dsRNA analogues.

Preferred examples of ribose-modified ribonucleotides are analogues wherein the 2′-OH group is replaced by a group selected from H, OR, R, halo, SH, SR, NH₂, NHR, NR₂ or CN with R being C₁-C₆ alkyl, alkenyl or alkynyl and halo being F, Cl, Br or I. It is clear in the context of the present invention, that the term “modified ribonucleoside triphosphate” or “modified ribonucleotide” also includes 2′-deoxy derivatives which may at several instances also be termed “deoxynucleotides”.

Typical examples of such ribonucleotide analogues with a modified ribose at the 2′ position include 5-aminoallyl-uridine , 2′-amino-2′-deoxy-uridine, 2′-azido-2′-deoxy-uridine, 2′-fluoro-2′-deoxy-guanosine and 2′-O-methyl-5-methyl-uridine.

Examples of ribonucleotides leading to a phosphate backbone modification in the desired dsRNA product are phosphothioate analogues.

According to the present invention, the at least one modified ribonucleotide may also be selected from analogues having a chemical modification at the base moiety. Examples of such analogues include, 6-aza-uridine, 8-aza-adenosine, 5-bromo-uridine, 7-deaza-adenosine, 7-deaza-guanosine, N⁶-methyl-adenosine, 5-methyl-cytidine, pseudo-uridine, and 4-thio-uridine.

The above and other chemically modified ribonucleoside triphosphates are commercially available, for example from Sigma-Aldrich Chemie GmbH, Munich, Germany or Trilink technologies, USA

Short DNA templates (e.g. as described above) are usually prepared by chemical synthesis. Other methods for providing the ssDNA(-containing) templates include enzymatic manipulations, for example reverse transcription of RNA and subsequent degradation of the RNA strand, cutting of larger dsDNA molecules by restriction enzyme(s) and subsequent strand separation by heat or chemical denaturation to form ssDNA and so on.

Preferred reaction volumes range from 20 to 200 microliter, preferably 50 to 100 microliter.

Typically, the buffer conditions and other conditions as outlined above are provided by mixing appropriate stock solutions (usually 5x or 10x concentrated), adding the RdRp, the template and double distilled or deionised water (which has been preferably made RNAse and/or DNAse free prior to use) to the desired final reaction volume.

As is evident from the present description, the invention generally relates to the use of the above-defined RdRps having DNA-dependent RNA polymerase activity for transcribing DNA into RNA.

The method of the present invention is also useful in techniques that usually start with (ss)DNA species and then turn to the RNA world. Such techniques typically require a transcription of the starting DNA material into RNA which is mostly carried out by use of transcriptases such as the T7 RNA polymerase (requiring a T7 specific promoter sequence). An example is the SELEX (systematic evolution of ligands by exponential enrichment) process for identifying and amplifying nucleotide sequences for binding to a certain target structure such as a protein or other biomolecule (see, in particular, WO-A-91/19813). Typically, a SELEX process starts with the chemical synthesis of a ssDNA library of sequences that contain at least a randomized sequence part. The ssDNA templates are then amplified by PCR resulting in a dsDNA library. The dsDNA molecules are transcribed into ssRNA usually by T7 RNA polymerase (the sequences therefore require a T7 promoter).The ssRNA library represents the starting library for the first round of the selection process: the RNA library (in an appropriate binding buffer) is loaded on a column containing the target structure coupled (typically with the aid of a spacer molecule) to an appropriate resin. In certain embodiments the RNA library is firstly contacted with the resin itself (e.g. present in a pre-column) not containing the target structure in order to eliminate sequences that bind to the resin itself. RNA molecules of the library that bind to the target structure coupled to the resin will be separated from the non-binding RNAs that appear in the flow-through. After elution of the binding RNA molecules from the column the thus-selected sequences are reverse transcribed into cDNA and amplified by PCR. The amplified DNA (representing the sequences that showed an affinity to the target structure in the first round of the selection process) are again transcribed into RNA which is then used for a further round of selection using the immobilised target structure. This process is typically reiterated yielding sequences with desirably high affinity to the target structure.

From the foregoing description of present the invention, it is evident that the method according to the invention can be used to avoid the steps of reverse transcription to cDNA and transcription (usually T7 transcription) of the PCR-amplified sequences into RNA in each round of the SELEX process. After preparation of the ssDNA starting library (which step will usually still be carried out as DNA molecules, since chemical synthesis of DNA is much less expensive than chemical synthesis of RNA) the ssDNA will be transcribed into RNA by the method of the invention (not requiring any specific promoter sequences that may interfere with the binding of the RNA molecules to the target structure). The selected sequences could then be amplified by the method of the invention serving directly as the starting material for the next SELEX round making the whole procedure more uncomplicated, cheaper and faster.

The terminal transferase activity of the RdRps as defined herein forms the basis of a further aspect of the present invention relating to a method for transferring one or more ribonucleotides to the 3′-end of single-stranded DNA (ssDNA) comprising the step of incubating the ssDNA in the presence of an RdRp as defined above and in the presence of rCTP or rGTP or rATP or rUTP under conditions such that said RdRp adds at least one of rC or rG or rA or rU to the 3′-end of said ssDNA.

As before, the ribonucleotide(s) added to the 3′-end of the ssDNA may be modified analogues (i.e. labelled as defined above and/or chemically modified as defined above).

Due to the terminal transferase activity of the RdRp as defined herein, the enzyme may add one or more nucleotides to the double-stranded transcription product even under DNA-dependent RNA polymerisation conditions or to a subsequent dsRNA product under RNA-dependent RNA polymerisation conditions (i.e. in the presence of all four rNTPs or analogues thereof) which depends on the specific conditions employed (buffer, temperature, incubation time, eventually present modified NTPs etc). If a resulting transcription product or dsRNA product happens to have a single stranded extension (at one or both sides of the double-stranded product) these may be eliminated by incubation with an enzyme degrading single-stranded polynucleotides, e.g. by S1 nuclease under conditions well known in the art.

The present invention is also of use for providing double-stranded RNA/DNA molecules having designed end regions. This aspect of the invention is, for example, applicable for providing dsRNA molecules of all types and lengths with designed end regions. An especially preferred application is the provision of correspondingly designed small dsRNA molecules, e.g. for RNAi applications.

Thus, the present invention further relates to a method for providing a double-stranded nucleic acid with at least one designed end using restriction digestion comprising the steps of:
(i) carrying out the transcription method as defined herein with the proviso that the template contains at least one recognition sequence of a restriction enzyme, preferably at least one recognition sequence of a restriction enzyme in 3′ of a selected sequence, wherein the at least one recognition sequence is composed of deoxynucleotides and the transcription is carried out either in the presence of a DNA primer matching the sequence of the at least one recognition sequence present in the template or in the absence of a primer; and
(ii) digesting the resulting double-stranded molecule with a restriction enzyme specific for the at least one recognition sequence.

The template may, of course, contain more than one recognition sequence of the same or different restriction enzymes. For example the template may contain a selected sequence (which may be of DNA or RNA or mixed DNA and RNA) flanked on the 5′ and the 3′ side by deoxyribonucleotides of a sequence corresponding to the recognition sequence(s) of the same or different restriction enzymes. Transcribing such a template as outlined above in the presence or absence of a primer matching the recognition sequence flanking the 3′-end of the selected sequence and digesting the resulting double-stranded product with the appropriate restriction enzyme(s) results in the double-stranded nucleic acid having ends that are determined by the cutting scheme of the employed restriction enzyme (generating blunt ends or ends having a 5′- or 3′ overhang). In the context of this method according to the invention it is clear that the recognition sequence(s) is/are typically selected such that these recognition sequences occur only at the desired locus/loci in the template

The template strand containing the at least one recognition sequence of a restriction enzyme may be prepared chemically or may be derived form chemically prepared and/or naturally occurring sequences and/or sequences derived from naturally occurring sequences by ligating corresponding sequences together such as by ligating an appropriate RNA sequence to a RNA/DNA or DNA sequence containing the restriction site by using an RNA ligase (e.g. T4 RNA ligase which is commercially available, e.g. from New England Biolabs, Ipswich, Mass., USA).

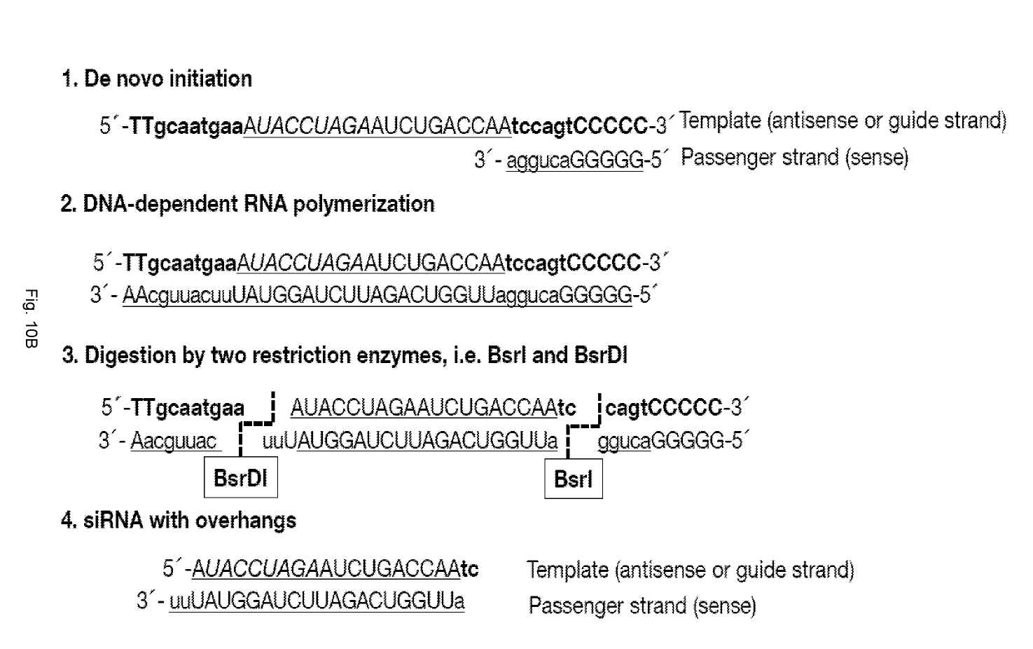

According to a first preferred embodiment of this method a template strand (which may be referred to as an “antisense” strand) is employed having a selected sequence (which may also be denoted as a “target” sequence) and containing, preferably directly following the 3′-end of the selected sequence, a recognition sequence of at least one restriction enzyme (or more recognition sequences of the same or other restriction enzymes) and having, according to preferred embodiments, at least one, more preferably at least three, even more preferably at least 5 C nucleotides at the very 3′-end of the template, wherein the C nucleotide(s) is/are either ribo- or deoxyribonucleotides and at least the nucleotides of the recognition sequence are deoxyribonucleotides. As disclosed above, C nucleotides can also be added to an appropriate template by using the terminal transferase activity of the RdRps as defined herein. The complete template may be composed of deoxynucleotides, but it may also contain ribonucleotides, for example, the complete or a part of the selected sequence may be composed of ribonucleotides. As mentioned before, the template may be prepared by chemical synthesis. It may also be prepared by preparing certain parts of the template and ligating these parts together. For example, one part such as the selected sequence or a part thereof may be composed of RNA which may be ligated using RNA ligase e.g. to the deoxynucleotide sequence comprising the recognition sequence of a restriction enzyme (and, optionally, containing further deoxyribonuncleotides and/or ribonucleotides as outlined above). A DNA primer matching the sequence of the recognition sequence and, if needed depending on the length and type of the recognition sequence, sufficient further nucleotides near or at the 3′-end of the template is hybridized under hybridisation conditions to the template. The template hybridised to the primer is then incubated under appropriate RNA synthesis conditions with an RdRp having DNA-dependent RNA polymerase activity as defined herein, producing a double-stranded molecule having a functional restriction site (which is preferably located directly 3′ with respect to the selected sequence of the template strand). The double-stranded molecule is then cut with an appropriate restriction enzyme resulting in the digestion products including the double-stranded nucleic acid containing the selected sequence of the antisense strand and the complementary sense strand and having at one end (with respect to the 3′-end of the antisense and the 5′-end of the sense strand, respectively) the design produced by the restriction enzyme (which may result in a blunt end, a 3′-overhang or a 5′-overhang). A specific example of such a method, in this case with respect to the provision of a siRNa having a 3′-overhang at the antisense (=guide) strand, is illustrated in FIG. 9A. The template (SEQ ID NO: 25), which may be provided by chemical synthesis, contains the selected sequence (here: the antisense (=guide) RNA strand of the siRNA to be produced) and directly in 3′ thereto the recognition sequence (deoxyribonucleotides) of a restriction enzyme (in this case a Bsr I site). The recognition sequence is followed by 5 dC. A ssDNA primer (SEQ ID NO: 26) is annealed to the template strand matching the recognition and the 5 dC nucleotides. The template hybridised to the primer is incubated under RNA polymerisations condition with an RdRp as defined herein which synthesises the complementary (sense or “passenger”) strand (SEQ ID NO: 27) of the siRNA. The resulting double-stranded product is incubated with the appropriate restriction enzyme (in this example Bsr I) producing the double-stranded siRNA (antisense or guide strand: SEQ ID NO: 28; sense or passenger strand: SEQ ID NO: 29) having the desired end, in the example a 3′-overhang at the antisense strand.

The above first embodiment of the method for providing double-stranded nucleic acids having defined ends can be modified utilising the capability of the RdRps having DNA-dependent RNA polymerase activity according to the invention to initiate RNA synthesis de novo (primer-independent RNA synthesis). In this second preferred embodiment of the inventive method for providing double-stranded RNA/DNA molecules having designed end regions, the template strand as outlined above with respect to the first embodiment, but having at least one C nucleotide at its 3′-end, is incubated under RNA polymerisation conditions in the absence of a primer with an RdRp having DNA-dependent RNA polymerase activity as defined herein. The resulting double-stranded molecule contains the recognition sequence(s) of the template strand (which may be denoted as the “antisense” strand) and the recognition sequence(s) as RNA in the complementary strand (“sense” strand). The double-stranded molecule is then digested with the appropriate restriction enzyme resulting in the digestion products including the double-stranded nucleic acid containing the selected sequence of the antisense strand and the complementary sense strand and having at one end (with respect to the 3′-end of the antisense and the 5′-end of the sense strand, respectively) the design produced by the restriction enzyme (which may result in a blunt end, a 3′-overhang or a 5′-overhang). A specific example of this embodiment, in this case with respect to the provision of a siRNa having a 3′-overhang at the antisense (=guide) strand, is illustrated in FIG. 9B. The template (SEQ ID NO: 25), which may be provided by chemical synthesis, contains the selected sequence (here: the antisense (=guide) RNA strand of the siRNA to be produced) and directly in 3′ thereto the recognition sequence (deoxyribonucleotides) of a restriction enzyme (in this case a Bsr I site). The recognition sequence is followed by 5 dC. The template, in the absence of a primer, is incubated under RNA polymerisation conditions with an RdRp as defined herein which starts RNA synthesis de novo (indicated by the short complementary RNA sequence (SEQ ID NO: 30) in part 1. of FIG. 9B) and synthesises the complementary (sense or passenger) strand (SEQ ID NO: 31) of the siRNA. The resulting double-stranded product is incubated with the appropriate restriction enzyme (in this example Bsr I) producing the double-stranded siRNA (antisense or guide strand: SEQ ID NO: 28; sense or passenger strand: SEQ ID NO: 29) having the desired end, in the example a 3′-overhang at the antisense strand.

According to a third preferred aspect of the method for providing double-stranded nucleic acids having defined ends as disclosed herein the resulting product contains a defined end at both ends of the double-stranded product. A template as outlined before for the first embodiment may also contain a deoxyribonucleotide sequence consisting of or containing the recognition sequence of at least one restriction enzyme (or containing more than one recognition sequence of the same or different restriction enzymes) near or directly 5′ to the selected sequence. As mentioned before, such templates may either be chemically synthesized or they may be assembled from chemically synthesized parts or from naturally occurring sequences or sequences derived from naturally sequences, e.g. by restriction or other manipulations known in the art, and ligating corresponding parts together such as by RNA ligase (e.g. T4 RNA Ligase), if sequences consisting or containing RNA (for example the selected sequence) and DNA (in particular the sequences containing the recognition sequence(s)) are to be assembled. As already outlined above for the first embodiment, a DNA primer matching the sequence of the one or more recognition sequence(s) present in the 3′ direction from the selected sequence and, if needed depending on the length and type of the recognition sequence, sufficient further nucleotides near or at the 3′end of the template is hybridized under hybridisation conditions to the template. The template hybridised to the primer is then incubated under appropriate RNA synthesis conditions with an RdRp having DNA-dependent RNA polymerase activity as defined herein, producing a double-stranded molecule having at least one restriction site on both sides of the selected sequence. The double-stranded product is then digested with the appropriate restriction enzyme(s) resulting in the digestion products including the double-stranded nucleic acid containing the selected sequence of the antisense strand and the complementary sense strand and having at both ends the design produced by the restriction enzyme(s) which may result on both sides of the selected sequence in a blunt end, a 3′-overhang or a 5′-overhang, respectively. A specific example of such a method, in this case with respect to the provision of a siRNA having designed ends on both sides of the dsRNA molecule, for example a 3′-overhang both at the antisense (=guide) strand and the sense (=passenger) strand, is illustrated in FIG. 10A. The template (SEQ ID: NO: 32) contains the selected sequence (here: the antisense (=guide) RNA strand of the siRNA to be produced). The template further contains each directly in 5′ and in 3′ to the selected sequence a recognition sequence (deoxyribonucleotides) of a restriction enzyme (in this case a Bsr I site on the 3′ side and a BsrD I site on the 5′ side). The recognition sequence on the 3′ side is followed by 5 dC. A ssDNA primer (SEQ ID NO: 26) is annealed to the template strand matching the recognition sequence at the 3′ side of the selected sequence and the 5 dC nucleotides. The template hybridised to the primer is incubated under RNA polymerisation conditions with an RdRp as defined herein which synthesises the complementary (sense or passenger) strand (SEQ ID NO: 33) of the siRNA. The resulting double-stranded product is incubated with the appropriate restriction enzymes (in this example Bsr I and BsrD I) producing the double-stranded siRNA (antisense or guide strand: SEQ ID NO: 28; sense or passenger strand: SEQ ID NO: 34) having the desired end regions generated by the restriction enzymes, in the example shown in FIG. 10A a 3′-overhang at the antisense strand and a 3′-overhang at the sense strand.

The above third embodiment of the method for providing double-stranded nucleic acids having defined ends can also be modified utilising the capability of the RdRps having DNA-dependent RNA polymerase activity according to the invention to initiate RNA synthesis de novo (primer-independent RNA synthesis). Thus, according to a fourth preferred embodiment of the inventive method for providing double-stranded RNA/DNA molecules having designed end regions, the template strand as outlined above with respect to the third embodiment, but having at least one C nucleotide at its 3′-end, is directly incubated under RNA polymerisation conditions with an RdRp having DNA-dependent RNA polymerase activity as defined herein. The resulting double-stranded molecule contains the recognition sequences of the template strand (which may be denoted as the “antisense” strand) located 5′ and 3′ to the selected sequence and the recognition sequences as RNA in the complementary strand (“sense” strand). The double stranded molecule is then digested with the appropriate restriction enzyme(s) resulting in the digestion products including the double-stranded nucleic acid containing the selected sequence of the antisense strand and the complementary sense strand and having at both ends the design produced by the restriction enzyme (which may result in a blunt end, a 3′-overhang or a 5′-overhang at each side). A specific example of this embodiment, in this case with respect to the provision of a siRNA having designed ends, for example having 3′-overhangs at the antisense (=guide) strand and the sense (=passenger strand), is illustrated in FIG. 10B. The template (SEQ ID NO: 32) contains the selected sequence (here: the antisense (=guide) RNA strand of the siRNA to be produced) and directly in 5′ and 3′ thereto the recognition sequences (deoxyribonucleotides) of two differendt restriction enzymes (in this example a Bsr I site at the 3′ end and a BsrD I site at the 5′ end). The recognition sequence at the 3′ end is followed by 5 dC. The template, in the absence of a primer, is incubated under RNA polymerisation conditions with an RdRp as defined herein which starts RNA synthesis de novo (indicated by the short complementary RNA sequence (SEQ ID NO: 30) in part 1. of FIG. 10B) and synthesises the complementary (sense or passenger) strand (SEQ ID NO: 35) of the siRNA. The resulting double-stranded product is incubated with the appropriate restriction enzymes (in this example Bsr I and BsrD I) producing the double-stranded siRNA (antisense or guide strand: SEQ ID NO: 28; sense or passenger strand: SEQ ID NO: 34) having defined ends, in the example shown in FIG. 10B a 3′-overhang at the antisense strand and a 3′ overhang at the sense strand.

Further subject matter of the present invention constitutes a kit for
at least one RdRp as defined herein;
rATP, rCTP, rGTP and rUTP which may be optionally modified (labelled and/or chemically modified as described above);
a buffer for providing conditions sufficient for DNA-dependent RNA synthesis by the RdRp;
a single-stranded polynucleotide control template of predetermined nucleotide sequence comprising at least a segment of DNA, preferably consisting of DNA, and having at least one C nucleotide (preferably at least one rC), more preferably at least 3 C nucleotides (e.g. 5 C nucleotides), at its 3′-end;
optionally, stop solution (preferred examples are desribed above);
optionally, primer (such as those as described above).

The figures show:

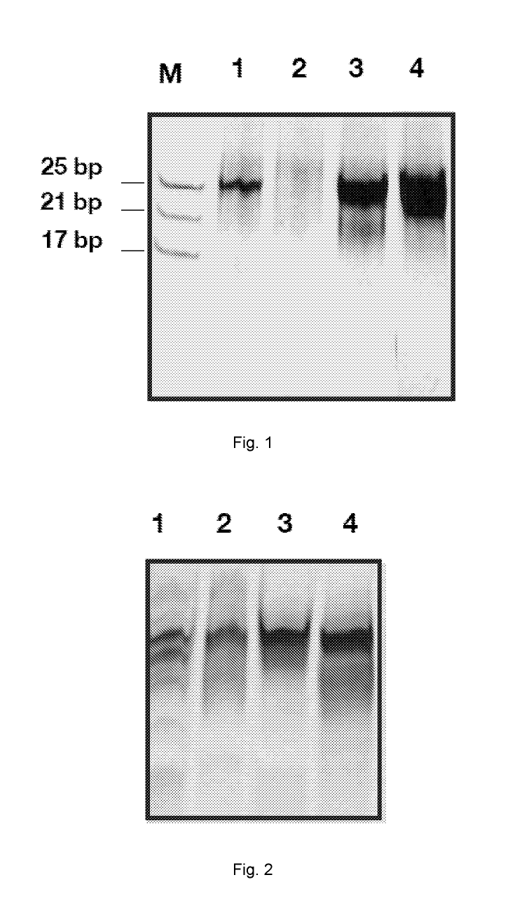

FIG. 1 shows a photograph of a polyacrylamide gel after electrophoretic separation of reaction mixtures for analysing the primer-independent de novo RNA synthesis and generation of a DNA/RNA double strand by a sapovirus RdRp on different ssDNA-containing templates. The sapovirus RdRp (SEQ ID NO: 11) was incubated with the following templates: lane 1: ssDNA template 5′-ATACCTAGAATCTGACCAACCCCC-3′ (SEQ ID NO: 15); lane 2: ssDNA template 5′-ATACCTAGAATCTGACCAA-3′ (SEQ ID NO: 16), i.e. the same sequence as in lane 1 but missing the (dC)₅ stretch at the 3′-end; lane 3: ssDNA template 5′-ATACCTAGAATCTGACCAArCrCrCrCrC-3′ (SEQ ID NO: 17), i.e. again the same nucleotide sequence but bearing a stretch of five C ribonucleotides at the 3′-end.; 4: ssRNA template 5′-AUACCUAGAAUCUGACCAACCCCC-3′ (SEQ ID NO: 18) serving as a positive control; lane M: dsRNA marker corresponding to a length of 17 bp, 21 bp and 25 bp, as indicated. A double-stranded product band of 24 bp is visible in lanes 1, 3 and 4.

FIG. 2 shows a further photograph of a polyacrylamide gel after electrophoretic separation of reaction mixtures demonstrating that a sapovirus RdRp is capable of initiating RNA synthesis on DNA templates de novo in a primer-independent manner leading to a double-stranded DNA/RNA product. The sapovirus RdRp (SEQ ID NO: 11) was incubated with a ssDNA template (5′-ATACCTAGAATCTGACCAACCCCC-3′, SEQ ID NO: 15; lane 1). The resulting product was incubated with S1 nuclease (lane 2). The sapovirus RdRp was also incubated with a ssDNA template bearing a (rC)₅ stretch at its 3′-end (5′-ATACCTAGAATCTGACCAArCrCrCrCrC-3′, SEQ ID NO: 17; lane 3). Also the product of this reaction was incubated with S1 nuclease (lane 4). The product band of 24 bp remains visible after incubation with S1 nuclease demonstrating the double-stranded nature of the sapovirus RdRp reaction product.

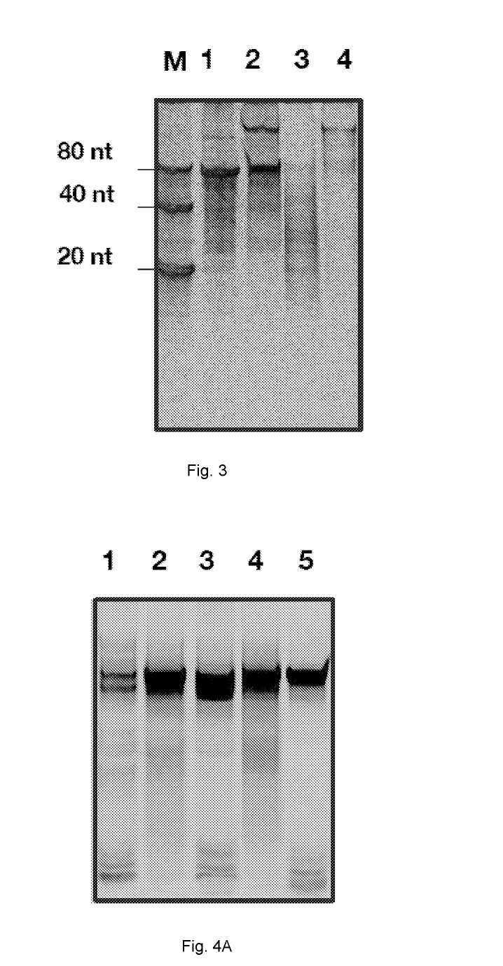

FIG. 3 shows a further photograph of a polyacrylamide gel after electrophoretic separation of reaction mixtures indicating that a lagovirus RdRp initiates RNA synthesis on DNA templates de novo in a primer-independent manner leading to a double-stranded DNA/RNA product. The band in lane 1 shows the ssDNA template (5′-ATACCTAGAATGTGACCAAATACCTAGAATCTGACCAACGAAAAAAAAAAUAA GCACGAAGCTCAGAGTCCCCC-3′; SEQ ID NO: 19) alone (without incubation with the lagovirus RdRp). The lagovirus RdRp (SEQ ID NO: 14) was incubated with the ssDNA template resulting in a band of lower electrophoretic mobility (lane 2). The ssDNA template (lane 1) or the resulting product (lane 2), respectively, was incubated with S1 nuclease. The single-stranded DNA template is completely digested by the S1 nuclease (lane 3). In contrast, no digestion of the product band is observed when incubated with S1 nuclease (lane 4), indicating the double-stranded stranded nature of the transcription product. M: DNA marker corresponding to single-stranded DNA of 80 nt, 40 nt and 20 nt in length, as indicated.

FIG. 4A shows a further photograph of a polyacrylamide gel after electrophoretic separation of reaction mixtures demonstrating that a sapovirus RdRp initiates RNA Synthesis on DNA templates de novo in a primer-independent manner and incorporates 2′-fluoro-GMP leading to a double-stranded DNA/RNA product. The sapovirus RdRp (SEQ ID NO: 11) was incubated with a single-stranded DNA template (lane 1; 5′-ATACCTAGAATCTGACCAACCCCC-3′, SEQ ID NO: 15) or a DNA template of the same sequence but bearing a (rC)₅ sequence motive at the 3′-terminus (lane 3; 5′-ATACCTAGAATCTGACCAA(rCrCrCrCrC-3′, SEQ ID NO: 17). As a positive control (lane 5), the sapovirus RdRp was incubated with a single-stranded RNA (5′-AUACCUAGAAUCUGACCAACCCCC-3′; SEQ ID NO: 18) displaying the same sequence as the single-stranded DNA. Incubation of the sapovirus RdRp was either carried out in the presence of rATP, rCTP, rUTP and rGTP (lanes 1 and 3) or in the presence of rATP, rCTP, rUTP and 2′-fluoro-GTP (lanes 2 and 4). A product band is visible when the sapovirus RdRp is incubated with the ssDNA template in the presence of unmodified rNTPs as well as in the presence of the modified GTP analogue.

FIG. 4B shows a further photograph of a polyacrylamide gel after electrophoretic separation of reaction mixtures demonstrating that a sapovirus RdRp initiates RNA Synthesis on DNA templates de novo in a primer-independent manner and incorporates α-thio-GMP leading to a double-stranded DNA/RNA product. The sapovirus RdRp (SEQ ID NO: 11) was incubated with a single-stranded DNA template (lane 1; 5′-ATACCTAGAATCTGACCAACCCCC-3′, SEQ ID NO: 15) or a ssDNA template of the same sequence but bearing a (rC)₅ sequence motive at the 3′-terminus (lane 2; 5′-ATACCTAGAATCTGACCAArCrCrCrCrC, SEQ ID NO: 17). As a positive control (lane 3), the sapovirus RdRp was incubated with a single-stranded RNA template displaying the same sequence as the single-stranded DNA (5′-AUACCUAGAAUCUGACCAACCCCC-3′, SEQ ID NO: 18). The reaction mix contained rATP, rUTP, rCTP and α-thio-GTP in the reactions of lanes 1 and 2, and contained rATP, rUTP, rCTP and unmodified rGTP in the control reaction of lane 3. A product band is visible in all three lanes.

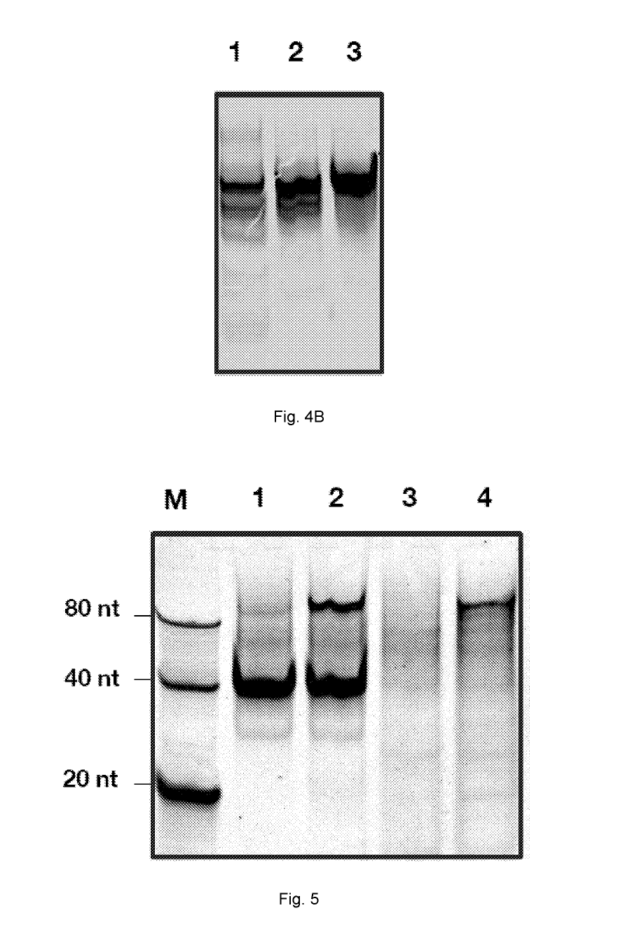

FIG. 5 shows a further photograph of a polyacrylamide gel after electrophoretic separation of reaction mixtures demonstrating that a vesivirus RdRp initiates RNA synthesis on certain DNA templates in a primer-dependent manner leading to a double-stranded DNA/RNA product. The vesivirus RdRp (SEQ ID NO: 13) was incubated with a single-stranded DNA template (lane 1; 5′-TTGCAATGAAATACCTAGAATCTGACCAATCCAGTAAAA-3′, SEQ ID NO: 19), or with the same DNA template hybridized to a DNA oligonucleotide primer (5′-TTTTACTGGA-3′; SEQ ID NO: 20) displaying a sequence complementary to the 3′-end of the DNA template (lane 2). In the absence of the primer, no product band is visible (lane 1) indicating that the DNA template is not transcribed. In contrast thereto, when the DNA template is hybridized to a primer, a product is generated (lane 2).The DNA template alone (lane 3) and the resulting transcription product (lane 4) were incubated with S1 nuclease. The single-stranded DNA template is completely digested by the S1 nuclease (lane 3). In contrast, no digestion of the transcription product is observed when incubated with S1 nuclease (lane 4), indicating the double-stranded nature of the transcription product. M: DNA marker corresponding to single-stranded DNA of 80 nt, 40 nt and 20 nt in length, as indicated.

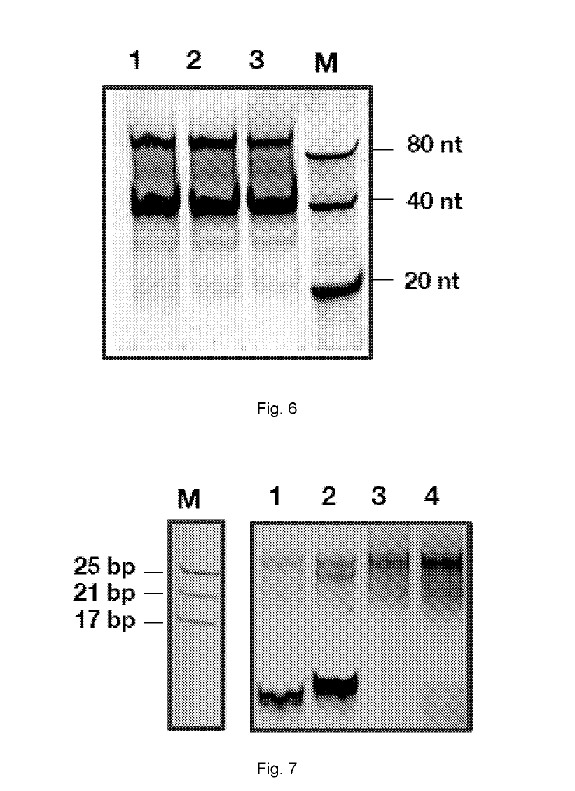

FIG. 6 shows a further photograph of a polyacrylamide gel after electrophoretic separation of reaction mixtures demonstrating that a vesivirus RdRp initiates RNA synthesis on certain DNA templates in a primer-dependent manner and incorporates α-thio-GMP or 2′-fluoro-GMP leading to a double-stranded DNA/RNA product. The vesivirus RdRp (SEQ ID NO: 13) was incubated with a single-stranded DNA template (lane 1; 5′-TTGCAATGAAATACCTAGAATCTGACCAATCCAGTAAAA-3′, SEQ ID NO: 19) hybridized to a DNA oligonucleotide primer (5′-TTTTACTGGA-3′, SEQ ID NO: 20) displaying a sequence complementary to the 3′-end of the DNA template. The reaction mix contained rATP, rUtP, rCTP and (unmodified) rGTP (lane 1) or rATP, rUtP, rCTP and α-thio-GTP (lane 2) or rATP, rUtP, rCTP and 2′-fluoro-GTP (lane 3). A product band with a lower electrophoretic mobility than a single-stranded DNA marker of 80 nt is visible in all reactions. M: DNA marker corresponding to single-stranded DNA of 80 nt, 40 nt and 20 nt in length, as indicated.

FIG. 7 shows a further photograph of a polyacrylamide gel after electrophoretic separation of reaction mixtures demonstrating the primer-independent de novo initiation of RNA synthesis on a DNA template onto which several C ribonucleotides have been added by the terminal transferase activity of the sapovirus RdRp. A sapovirus RdRp (SEQ ID: 11) was incubated with a single-stranded DNA template not bearing (lane 1; 5′-CCCCCTTGGTCAGATTCTAGGTAT-3′, SEQ ID NO: 21) or bearing a single (rC) nucleotide at the 3′-terminus (lane 2; 5′-CCCCCTTGGTCAGATTCTAGGTAT(rC)-3′, SEQ ID NO: 22). In the absence of an rC as the ultimate nucleotide at the 3′-end of the DNA template, no primer-independent initiation of RNA synthesis occurs, and no DNA/RNA double strand is produced. In the presence of a single (rC), de novo (i.e. primer-independent) initiation of RNA synthesis occurs, leading to DNA/RNA double strand (although the transcription efficiency is decreased as compared to experiments wherein the template contains more C nucleotides at the 3′-end). In a further experimental set-up, the sapovirus RdRp was first used as a terminal transferase to append a poly(C)-motive at the 3′-end of the ssDNA template. Therefore, the sapovirus RdRp was incubated with the DNA template not bearing (lane 3; 5′-CCCCCTTGGTCAGATTCTAGGTAT-3′, SEQ ID NO: 21) or bearing a single (rC) nucleotide at the 3′-terminus (lane 4; 5′-CCCCCTTGGTCAGATTCTAGGTAT(rC)-3′, SEQ ID NO: 22) firstly in presence of rCTP as the only rNTP and afterwards in the presence of all four rNTPs. As demonstrated by the band in lane 3 and lane 4 co-migrating with a 25 bp dsRNA marker, a DNA/RNA-double stranded product is generated after adding a poly(rC)-motive to the 3′-end of the ssDNA template by the terminal transferase activity of the sapovirus RdRp. M: dsRNA marker corresponding to a length of 17 bp, 21 bp and 25 bp, as indicated.

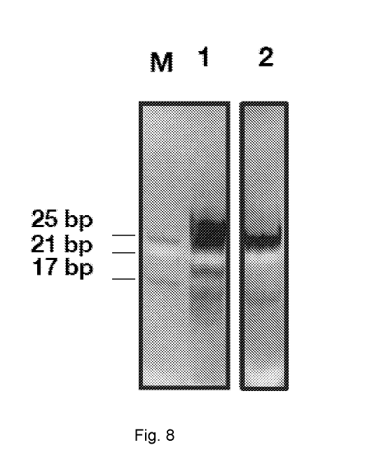

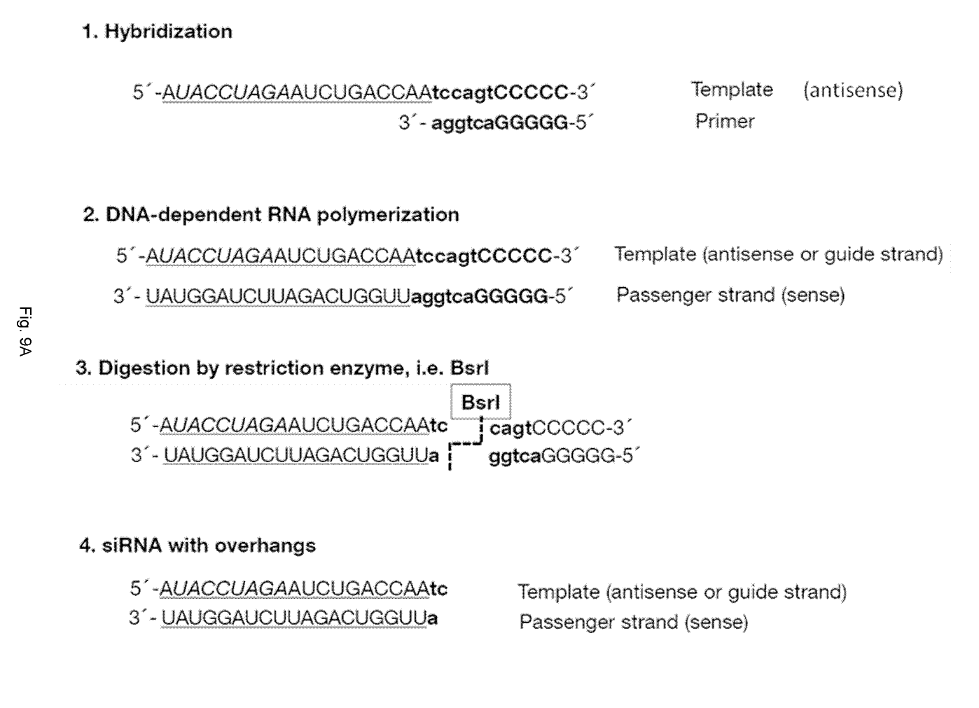

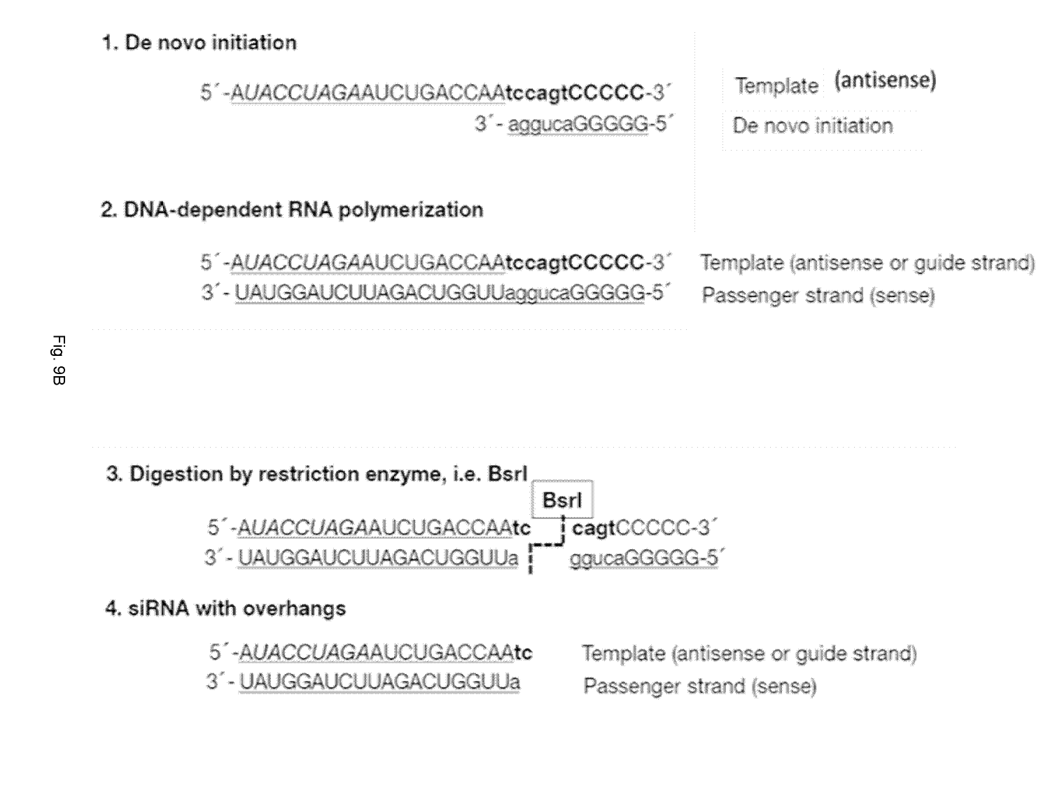

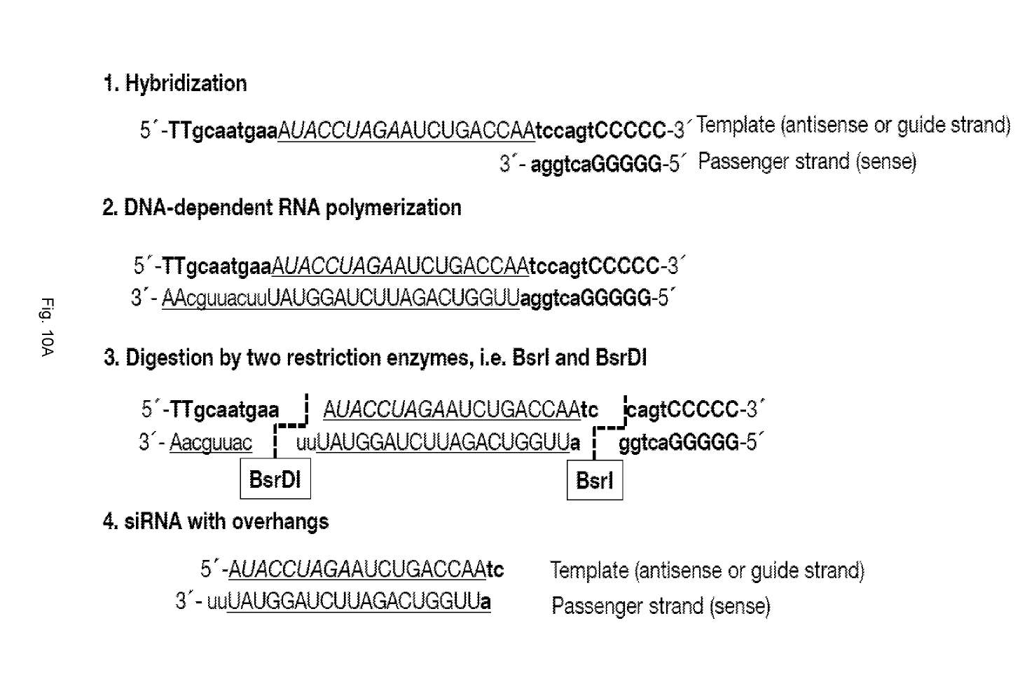

FIG. 8 shows a further photograph of a polyacrylamide gel after electrophoretic separation of reaction mixtures demonstrating the primer-independent de novo initiation of RNA synthesis by a norovirus RdRp on a DNA-containing template. A norovirus RdRp (SEQ ID NO: 10) was incubated with a mixed RNA-DNA template bearing a (dC)₅ sequence motive at the 3′-terminus (5′-UAAGCACGAAGCUCAGAGUdCdCdCdCdC-3′, SEQ ID NO: 23; lane 1). As a positive control (lane 2), the norovirus RdRp was incubated with a single-stranded RNA having the same sequence (5′-UAAGCACGAAGCUCAGAGUCCCCC-3′, SEQ ID NO: 24) as the RNA-DNA template but containing only ribonucleotides. A product band of 24 bp is generated in both reactions. M: dsRNA marker corresponding to a length of 17 bp, 21 bp and 25 bp, as indicated.

FIG. 9A shows a schematic representation illustrating an embodiment of a method for providing a dsRNA, here a siRNA, having a 3′-overhang at one side of the dsRNA using primer-dependent DNA-dependent RNA synthesis according to the invention. Deoxyribonucleotides are in bold, ribonucleotides are underlined. Lower case letters indicate a recognition site for a restriction enzyme. Letters in italic indicate the seed region of the antisense (=guide) strand of an siRNA.

FIG. 9B shows a schematic representation illustrating an embogidment of a method for providing a dsRNA, here a siRNA, having a 3′-overhang at one side of the dsRNA using primer-independent DNA-dependent RNA synthesis according to the invention. Lower case letters indicate a recognition site for a restriction enzyme. Letters in italic indicate the seed region of the antisense (=guide) strand of an siRNA.

FIG. 10A shows a schematic representation illustrating an embodiment of a method for providing a dsRNA, here a siRNA, having 3′-overhangs at both sides of the dsRNA using primer-dependent DNA-dependent RNA synthesis according to the invention. Lower case letters indicate a recognition site for a restriction enzyme. Letters in italic indicate the seed region of the antisense (=guide) strand of an siRNA.

FIG. 10B shows a schematic representation illustrating an embodiment of a method for providing a dsRNA, here a siRNA, having 3′-overhangs at both sides of the dsRNA using primer-independent DNA-dependent RNA synthesis according to the invention. Lower case letters indicate a recognition site for a restriction enzyme. Letters in italic indicate the seed region of the antisense (=guide) strand of an siRNA.

The present invention is further illustrated by the following non-limiting examples.

### EXAMPLES

### Example 1

### Primer-Independent de novo Initiation of RNA Synthesis and Generation of a DNA/RNA Double Strand by Sapovirus RdRp

A sapovirus RdRp (SEQ ID NO: 11) was incubated with a single-stranded DNA template bearing (5′-ATACCTAGAATCTGACCAACCCCC-3′, SEQ ID NO: 15) or not bearing (5′-ATACCTAGAATCTGACCAA-3′, SEQ ID NO: 16) a (dC)₅ sequence motive at the 3′-terminus or bearing a (rC)₅ sequence motive at the 3′-terminus (5′-ATACCTAGAATCTGACCAArCrCrCrCrC, SEQ ID NO: 17). As a control, the calicivirus RdRp was incubated with a single-stranded RNA (5′-AUACCUAGAAUCUGACCAACCCCC-3′, SEQ ID NO: 18) displaying the same sequence as the single-stranded DNA template of SEQ ID NO: 15. In the absence of a primer, the sapovirus RdRp generates a DNA/RNA double strand using a single-stranded DNA as the template containing a C nucleotide (rC or dC) at the 3′-end (FIG. 1, lanes 1 and 3). All reactions were performed in a total volume of 25 μl at 30° C. for 1 h. The reaction mix contained 1 μg template, 7.5 μM RdRp, 0.4 mM of each rATP, rCTP, rUTP, and 2 mM rGTP, 5 μl reaction buffer (HEPES 250 mM, MnCl₂ 25 mM, DTT 5 mM, pH 7.6), and RNAse-DNAse free water to a total volume of 25 μl. The products were visualized by ethidium bromide staining of a native 20% polyacrylamide gel after electrophoresis (FIG. 1).

### Example 2

### The Product Synthesized by Sapovirus RdRp on a ssDNA Template is Resistant to Digestion by S1 Nuclease

The sapovirus RdRp (SEQ ID NO: 11) used in Example 1 was incubated with a single-stranded DNA template (5′-ATACCTAGAATCTGACCAACCCCC-3′, SEQ ID NO: 15). The resulting product (FIG. 2, lane 1) was incubated with S1 nuclease. No digestion of the product is observed after incubation with S1 nuclease (FIG. 2, lane 2) indicating the double-stranded nature of the product generated with the sapovirus RdRp on the ssDNA template. In a further experimental set-up, the sapovirus RdRp (SEQ ID NO: 11) was incubated with a DNA template of the same sequence but bearing a (rC)₅ sequence motive at the 3′-terminus (5′-ATACCTAGAATCTGACCAA rCrCrCrCrC, SEQ ID NO: 17). The resulting product (FIG. 2, lane 3) was also incubated with S1 nuclease. Again, no digestion of the product is observed after incubation with S1 nuclease (FIG. 2, lane 4) indicating the double-stranded nature of the transcription product. All reactions were performed in a total volume of 25 μl a t 30° C. for 1 h. The reaction mix contained 1 μg template, 7.5 μM RdRp, 0.4 mM of each rATP, rCTP, rUTP, and 2 mM rGTP, 5 μl reaction buffer (HEPES 250 mM, MnCl₂ 25 mM, DTT 5 mM, pH 7.6), and RNAse-DNAse free water to a total volume of 25 μl. For S1 nuclease digestion, S1 nuclease (250 U) was added to the reaction and the reaction mix incubated for 1 h at 30° C. The products of the RNA synthesis and the S1 nuclease digestion were visualized by ethidium bromide staining of a native 20% polyacrylamide gel after electrophoresis (FIG. 2).

### Example 3

### Primer-Independent de novo Initiation of RNA Synthesis and Generation of a S1 Nuclease-Resistant DNA/RNA Double Strand by lagovirus RdRp

A lagovirus RdRp (SEQ ID: 13) was incubated with a single-stranded DNA template (5′-ATACCTAGAATGTGACCAAATACCTAGAATCTGACCAACGAAAAAAAAAAUAAGCACGAAGCTCAGAGTCCCCC-3′, SEQ ID NO: 19) (see FIG. 3, lane 2). The DNA template alone and the resulting transcription product, respectively, was incubated with S1 nuclease. The single-stranded DNA template is completely digested by S1 nuclease (Fig. Lane 3). In contrast thereto, no digestion of the transcription product is observed after incubation with S1 nuclease (FIG. 3, lane 4) indicating the double-stranded nature of the product of transcription by the lagovirus RdRp. All reactions were performed in a total volume of 25 μl at 30° C. for 1 h. The reaction mix contained 2 μg template, 7.5 μM RdRp, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAsefree water to a total volume of 25 μl. For S1 nuclease digestion, S1 nuclease (250 U) was added to the reaction and the reaction mix incubated for 1 h at 30° C. The products of the RNA synthesis and S1 digestion, respectively, were visualized by ethidium bromide staining of a native 20% polyacrylamide gel after electrophoresis (FIG. 3).

### Example 4

### Incorporation of Modified Nucleotides During Transcription of ssDNA Templates by Sapovirus RdRp

A sapovirus RdRp (SEQ ID NO: 11) was incubated with a DNA template (5′-ATACCTAGAATCTGACCAACCCCC-3′; SEQ ID NO: 15; see FIG. 4A, lane 1) or a DNA template bearing a (rC)₅ sequence motive at the 3′-terminus (5′-ATACCTAGAATCTGACCAA (rCrCrCrCrC, SEQ ID NO: 17; see FIG. 4A, lane 3). As a control the sapovirus RdRp was incubated with a single-stranded RNA template displaying the same sequence as the single-stranded DNA (5′-AUACCUAGAAUCUGACCAACCCCC-3′, SEQ ID NO: 18). All reactions were performed in a total volume of 25 μl at 30° C. for 1 h. The reaction mix contained 1 μg template, 7.5 μM RdRp, 0.4 mM of each rATP, rCTP, rUTP, and either unmodified rGTP (see FIG. 4A, lanes 1 and 3) or 2′-fluoro-GTP (FIG. 4A, lanes 2 and 4), 5 μl reaction buffer (HEPES 250 mM, MnCl₂ 25 mM, DTT 5 mM, pH 7.6), and RNAse-DNAsefree water to a total volume of 25 μl. The reaction products were visualized by ethidium bromide staining of a native 20% polyacrylamide gel after electrophoresis (FIG. 4A) showing that a transcription product was produced by the sapovirus RdRp both in presence of unmodified and 2′-fluoro-GTP.

In a further experiment, the sapovirus RdRp (SEQ ID NO: 11) was incubated with the same ssDNA template (5′-ATACCTAGAATCTGACCAACCCCC-3′, SEQ ID NO: 15; see FIG. 4B, lane 1) or the DNA template bearing a (rC)₅ sequence motive at the 3′-terminus (5′-ATACCTAGAATCTGACCAA (rCrCrCrCrC, SEQ ID NO: 17; see FIG. 4B lane 2) as before. As a control, the sapovirus RdRp was incubated with a single-stranded RNA displaying the same sequence as the single-stranded DNA (5′-AUACCUAGAAUCUGACCAACCCCC-3′, SEQ ID NO: 18; see FIG. 4B, lane 3). All reactions were performed in a total volume of 25 μl at 30° C. for 1 h. The reaction mix contained 1 μg template, 7.5 μM RdRp, 0.4 mM of each rATP, rCTP, rUTP, and either α-thio-GTP (FIG. 4B, lanes 1 and 2) or unmodified rGTP (FIG. 4B, lane 3), 5 μl reaction buffer (HEPES 250 mM, MnCl₂ 25 mM, DTT 5 mM, pH 7.6), and RNAse-DNAse free water to a total volume of 25 μl. The products of the RNA synthesis were visualized by ethidium bromide staining of a native 20% polyacrylamide gel after electrophoresis (FIG. 4B) showing that a transcription product was produced by the sapovirus RdRp both in presence of unmodified rGTP and α-thio-GTP.

### Example 5

### RNA Synthesis by Vesivirus RdRp on ssDNA Templates in the Presence of a Primer

A vesivirus RdRp (SEQ ID NO: 13) was incubated with a single-stranded DNA template (5′-TTGCAATGAAATACCTAGAATCTGACCAATCCAGTAAAA-3′, SEQ ID NO: 19; see FIG. 5, lane 1), or with the same ssDNA template hybridized to a DNA oligonucleotide primer (5′-TTTTACTGGA-3′, SEQ ID NO: 20; see FIG. 5, lane 2) displaying a sequence complementary to the 3′-end of the ssDNA template. In the absence of a primer, the ssDNA template having an A nucleotide at its 3′-end is not transcribed (FIG. 5, lane 1). However, if the ssDNA is hybridized to a primer, a transcription product is generated (FIG. 5, lane 2).The DNA template alone and the product resulting from transcription in the presence of a primer were incubated with S1 nuclease. The single-stranded DNA template is completely digested by the S1 nuclease (FIG. 5, lane 3) whereas no digestion of the product generated by transcription with the vesivirus RdRp in the presence of a primer is observed after incubation with S1 nuclease (FIG. 5, lane 4) indicating the double-stranded nature of the transcription product. All reactions were performed in a total volume of 25 μl at 30° C. for 1 h. The reaction mix contained of 2 μg template, 7.5 μM RdRp, 0.4 mM of each rATP, rCTP, rUTP, and 2 mM rGTP, 5 μl reaction buffer (HEPES 250 mM, MnCl₂ 25 mM, DTT 5 mM, pH 7.6), and RNAse-DNAsefree water to a total volume of 25 μl. Primer was added at a concentration of 0.25 μg/μl in the hybridization reaction. The products of the RNA synthesis were visualized by ethidium bromide staining of a native 20% polyacrylamide gel after electrophoresis (FIG. 5).

### Example 6

### Incorporation of Modified Nucleotides During Transcription of ssDNA Templates by Vesivirus RdRp in the Presence of a Primer

A vesivirus RdRp (SEQ ID NO: 13) was incubated with a single-stranded DNA template (5′-TTGCAATGAAATACCTAGAATCTGACCAATCCAGTAAAA-3′, SEQ ID NO: 19) hybridized to a DNA oligonucleotide primer (5′-TTTTACTGGA-3′, SEQ ID NO: 29) displaying sequence complementary to the 3′-end of the ssDNA template. All reactions were performed in a total volume of 25 μl at 30° C. for 1 h. The reaction mix contained 2 μg template, 7.5 μM RdRp, 0.4 mM of each rATP, rCTP, rUTP, and either unmodified rGTP (FIG. 6, lane 1) or 2′-fluoro-GTP (FIG. 6, lane 2) or α-thio-GTP (FIG. 6, lane 3), 5 μl reaction buffer (HEPES 250 mM, MnCl₂ 25 mM, DTT 5 mM, pH 7.6), and RNAse-DNAse free water to a total volume of 25 μl. The products were visualized by ethidium bromide staining of a native 20% polyacrylamide gel after electrophoresis (FIG. 6). A product band is visible in the presence of unmodified GTP as well as both 2-fluoro-GTP and α-thio-GTP (FIG. 6, lanes 1 to 3).

### Example 7

### Primer-Independent de novo Initiation of RNA Synthesis and Generation of a DNA/RNA-Double Strand After Adding C Nucleotides to a ssDNA Template Using the DNA-Dependent RNA Polymerase and Terminal Transferase Activities of Sapovirus RdRp

A sapovirus RdRp (SEQ ID NO: 11) was incubated with a single-stranded DNA template not bearing (5′-CCCCCTTGGTCAGATTCTAGGTAT-3′, SEQ ID NO. 20; see FIG. 7, lane 1) or bearing a single (rC) nucleotide at the 3′-terminus (5′-CCCCCTTGGTCAGATTCTAGGTAT(rC)-3′, SEQ ID NO: 22; see FIG. 7, lane 2). All reactions were performed in a total volume of 25 μl at 30° C. for 1 h. The reaction mix contained 1 μg template, 7.5 μM RdRp, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAse free water to a total volume of 25 μl. The products were visualized by ethidium bromide staining of a native 20% polyacrylamidegel after electrophoresis (FIG. 7, lanes 1 and 2). In the absence of an (rC) as the ultimate nucleotide at the 3′-end of the DNA template, no initiation of RNA synthesis occurs, and no DNA/RNA double strand is produced (FIG. 7, lane 1). In the presence of a single (rC) at the 3′-end of the ssDNA template, initiation of RNA synthesis occurs, leading to a DNA/RNA double strand (although the transcription efficiency is somewhat decreased as compared to experiments wherein the template contains more C nucleotides at the 3′-end)

In a further experimental set-up, the sapovirus RdRp was first used as a terminal transferase to append a poly(C)-motive at the 3′-end of the ssDNA template. Therefore, the sapovirusRdRp was first incubated with the same ssDNA templates as before in the presence of rCTP as the only nucleotide.

All reactions were performed in a total volume of 5 μl at 30° C. for 30 min. The terminal transferase reaction mix contained 1 μg template, 7.5 μM RdRp, 0.4 mMof CTP, 1 μl reaction buffer (HEPES 250 mM, MnCl₂ 25 mM, DTT 5 mM, pH 7.6), and RNAse-DNAsefree water to a total volume of 5 μl. In the next step (transcription of the template with added C nucleotides at the 3′-end), 5 μl of the previous reaction mix was incubated with the sapovirus RdRp in a total volume of 25 μl at 30° C. for 1 h. As observed in FIG. 6, lanes 3 and 4, a double stranded DNA/RNA product is generated after adding a poly(rC)-motive at the 3′-end of the DNA template by the terminal transferase activity of the sapovirus RdRp. The reaction mix for transcription by the sapovirus RdRp contained 1 μg template, 7.5 μM RdRp, 0.4 mM of each rATP, rCTP, rUTP, and 2 mMGTP, 5 μl reaction buffer (HEPES 250 mM, MnCl2 25 mM, DTT 5 mM, pH 7.6), and RNAse-DNAse free water to a total volume of 25 μl. The products were visualized by ethidium bromide staining of a native 20% polyacrylamide gel after electrophoresis (FIG. 7).

### Example 8

### Primer-Independent de novo Initiation of RNA Synthesis on a DNA Sequence and Generation of a DNA/RNA-Double Strand by Norovirus RdRp

A norovirus RdRp (SEQ ID NO: 10) was incubated with a mixed RNA-DNA template bearing a (dC)₅ sequence motive at the 3′-terminus (5′-UAAGCACGAAGCUCAGAGUdCdCdCdCdC-3′, SEQ ID NO: 23; see FIG. 8, lane 1). As a positive control (FIG. 8, lane 2), the norovirus RdRp was incubated with a single-stranded RNA having the same sequence (5′-UAAGCACGAAGCUCAGAGUCCCCC-3′, SEQ ID NO: 24) as the RNA-DNA template but containing only ribonucleotides. The norovirus RdRp generates a DNA/RNA double strand using the single-stranded template containing a DNA (dC)₅ sequence at the 3′-end (FIG. 8, lane 1).

All reactions were performed in a total volume of 25 μl at 30° C. for 1 h. The reaction mix contained 1 μg template, 7.5 μM RdRp, 0.4 mM of each rATP, rCTP, rUTP and rGTP, 5 μl reaction buffer (HEPES 250 mM, MnCl₂ 25 mM, DTT 5 mM, pH 7.6), and RNAse-DNAse free water to a total volume of 25 μl. The products were visualized by ethidium bromide staining of a native 20% polyacrylamide gel after electrophoresis (FIG. 8).

1. A method for transcribing a single-stranded polynucleotide template containing at least a segment of DNA into complementary RNA comprising the step of incubating said template with an RNA-dependent RNA polymerase (RdRp) having DNA-dependent RNA polymerase activity in the presence or absence of a primer hybridised to the single-stranded polynucleotide template under conditions such that said RdRp synthesizes an RNA strand complementary to said single-stranded polynucleotide template producing a double-stranded molecule comprising at least a segment of hybrid DNA/RNA,
wherein the RdRp having DNA-dependent RNA polymerase activity has a “right to hand conformation” and the amino acid sequence of said RdRp comprises a conserved arrangement of the following sequence motifs:
a.
(SEQ ID NO: 1)
XXDYS
b.
(SEQ ID NO: 2)
GXPSG
c.
(SEQ ID NO: 3)
YGDD
d.
(SEQ ID NO: 4)
XXYGL
e.
(SEQ ID NO: 5)
XXXXFLXRXX
wherein, if said template has a deoxy-T, deoxy-G or deoxy-A nucleotide at a 3′-end or if said template, the incubation step is carried out in the presence of a primer hybridised to the template.

2. The method of claim 1 wherein the RdRp having DNA-dependent RNA polymerase activity is an RdRp of a virus of the Caliciviridae family.

3. The method of claim 1 wherein the RdRp having DNA-dependent RNA polymerase activity is an RdRp of a noroviurs, sapovirus, vesivirus or lagovirus.

4. The method of claim 3 wherein the RdRp has an amino acid sequence selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

5. The method of claim 1 further comprising the step of (b) separating the double-stranded molecule comprising at least a segment of hybrid DNA/RNA into single strands producing a single-stranded RNA (ssRNA) and a single stranded molecule comprising at least a segment of single-stranded DNA (ssDNA).

6. The method of claim 5 wherein the step of separating the double-stranded molecule comprising at least a segment of hybrid DNA/RNA into single strands is performed by said RdRp.

7. The method of claim 5 further comprising the steps of:
(c) incubating the single strands obtained in step (b) with said RdRp under conditions such that the RdRp synthesizes an RNA strand complementary to each of said single strands to form double-stranded RNA (dsRNA) and a double-stranded molecule comprising at least a segment of hybrid DNA/RNA;
(d) optionally, separating the double-stranded products obtained in step (c) into single strands; and
(e) optionally, repeating steps (c) and (d) one or more times.

8. The method of claim 7 wherein, if performed, step (d) is carried out by said RdRp.

9. The method of claim 7 wherein, if steps (d) and (e) are performed, the method further comprises a final step of RNA synthesis by said RdRp.

10. The method of claim 1 wherein the single-stranded polynucleotide template has a segment of ssDNA at the 3′-end.

11. The method of claim 10 further comprising an initial step of incubating said single-stranded template with said RdRp and in the presence of rCTP as the only nucleotide under conditions so that said RdRp adds at least one rC residue to the 3′-end of the ssDNA or ssDNA segment, respectively.

12. The method of claim 1 wherein said RdRp incorporates at least one modified ribonucleotide during the step(s) of synthesizing a complementary RNA strand, the ribonucleotide comprising a ribose, a base attached to the ribose, and a phosphate moiety attached to the ribose.

13. The method of claim 12 wherein the at least one modified ribonucleotide contains a label for the detection of the double-stranded molecule produced by said RdRp.

14. The method of claim 13 wherein said label is selected from the group consisting of fluorophores, radioactive groups and partners of specific binding pairs.

15. The method of claim 13 wherein the at least one modified ribonucleotide is selected from the group consisting of 2′-O-methyl-cytidine, 2′-amino-2′-deoxy-uridine, 2′-azido-2′-deoxy-uridine, 2′-fluoro-2′-deoxy-guanosine, 2′-O-methyl-5-methyl-uridine-5′-triphosphate, 5-aminoallyl-uridine′, 6-aza-uridine, 8-aza-adenosine, 5-bromo-uridine, 7-deaza-adenosine, 7-deaza-guanosine, N⁶-methyl-adenosine, 5-methyl-cytidine, pseudo-uridine, 4-thio-uridine and phosphothioate analogues.

16. A method for transferring one or more ribonucleotides to a 3′-end of a single-stranded DNA (ssDNA) comprising the step of incubating the ssDNA in the presence of an RdRp and in the presence of an rNTP selected from the group consisting of rCTP, rGTP, rATP, rUTP, and a modified or labelled analogue thereof under conditions such that said RdRp adds at least one nucleotide selected from the group consisting of rC, rG, rA, rU, and a modified analogue thereof to the 3′-end of said ssDNA.

17. A method of using an RdRp as defined in claim 1 for the transcription of DNA into RNA.

18. A method for providing a double-stranded nucleic acid with at least one designed end using restriction digestion comprising the steps of:
(i) carrying out the method according to claim 1 wherein the template contains at least one recognition sequence of a restriction enzyme; and
(ii) digesting the resulting double-stranded molecule with the restriction enzyme specific for the at least one recognition sequence.

19. A kit for the transcription of DNA into RNA comprising:
a. an RdRp as defined in claim 1;
b. rATP, rCTP, rGTP and rUTP which may be optionally modified;
c. a buffer for providing conditions sufficient for DNA-dependent RNA synthesis by the RdRp;
d. a single-stranded polynucleotide control template of predetermined nucleotide sequence comprising at least a segment of DNA, preferably consisting of DNA, and having at least one C nucleotide, preferably at least 3 C nucleotides, at a 3′-end;
e. optionally, a stop solution;
f. optionally, a primer.

20. The kit of claim 19 wherein the control template has at least one C ribonucleotide at the 3′-end.

21. The method of claim 13 wherein the at least one ribonucleotide has a chemical modification at at least one structure selected from the group consisting of ribose, base and phosphate moiety.

22. The method of claim 18 , wherein at least one recognition sequence of a restriction enzyme is at a 3′-end of a selected sequence, wherein the at least one recognition sequence is composed of deoxynucleotides and the transcription is carried out in the presence of a DNA primer matching the sequence of the at least one recognition sequence present in the template.
